(19) United States
(12) Patent Application Publication
(10) Pub. No.: US 2016/0303154 A1
(43) Pub. Date: Oct. 20, 2016

(54) Topical Gel Composition Comprising Ivermectin

(71) Applicant: Gavis Pharmaceuticals, Somerset, NJ (US)

(72) Inventor: Bala Chandran Nayar, Somerset, NJ (US)

(73) Assignee: Gavis Pharmaceuticals, Somerset, NJ (US)

(21) Appl. No.: 14/687,786

(22) Filed: Apr. 15, 2015

(51) Int. Cl. A61K 31/7048; A61K 47/26; A61K 47/10; A61K 9/00; A61K 9/06

(52) U.S. Cl. CPC A61K 31/7048; A61K 9/0014; A61K 9/06; A61K 47/10; A61K 47/26

(57) ABSTRACT

A topical gel composition of ivermectin is provided. The topical gel composition contains a pharmaceutically acceptable glycol and a pharmaceutically acceptable gelling agent. The composition is essentially devoid of any alcohol.

### BACKGROUND OF THE INVENTION

(a) Field of the Invention

The present invention is directed to a topical gel composition of ivermectin. The composition is particularly in the form of an aqueous gel composition comprising a pharmaceutically acceptable glycol and a gelling agent. The invention is also directed to use of said topical gel composition of ivermectin for the treatment of rosacea, and particularly, for the treatment of inflammatory lesions of rosacea.

(b) Description of the Related Art

Ivermectin is a mixture of two compounds belonging to the class of avermectins, 5-O-demethyl-22,23-dihydroavermectin A₁ₐ and 5-O-demethyl-22,23-dihydroavermectin A_{1b}. They are also known as 22,23-dihydroavermectin B₁ₐ and 22,23-dihydroavermectin B_{1b}. Ivermectin is a mixture containing at least 90% 5-O-demethyl-22,23-dihydroavermectin A₁ₐ and less than 10% 5-O-demethyl-25-de(1-methylpropyl)-22,23-dihyro-25-(1-methylethyl) avermectin A₁ₐ, generally referred to as 22,23-dihydro avermectin B₁ₐ and B_{1b}, or H₂B₁ₐ and H₂B_{1b}, respectively. Ivermectin contains at least 80% of 22,23-dihydroavermectin B₁ₐ and less than 20% of 22,23-dihydroavermectin B_{1b}. The structural formulas are:

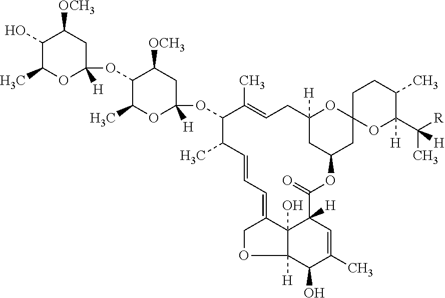

Component B₁ₐ R═C₂H₅ and Component B_{1b} R═CH₃

This active agent forms part of the class of avermectins, a group of macrocyclic lactones produced by the bacterium Streptomyces avermitilis (Reynolds J E F (Ed) (1993) Martindale, The Extra Pharmacopoeia. 29th Edition. Pharmaceutical Press, London). Avermectins include in particular ivermectin, invermectin, avermectin, abamectin, doramectin, eprinomectin and selamectin.

In the middle of the 1980s, ivermectin was presented as a broad-spectrum anti-parasitic medicinal product for veterinary use (W. C. Campbell, et al., (1983). Ivermectin: a potent new anti-parasitic agent, Science, 221, 823-828). It is effective against most common intestinal worms (except tapeworms), most acarids and some lice. It in particular exhibits considerable affinity for the glutamate-dependent chloride channels present in invertebrate nerve cells and muscle cells. Its binding to these channels promotes an increase in membrane permeability to chloride ions, resulting in hyperpolarization of the nerve or muscle cell. Neuromuscular paralysis which can lead to the death of certain parasites results therefrom. Ivermectin also interacts with other ligand-dependent chloride channels, such as those involving the neuromediator GABA (gamma-aminobutyric acid).

Ivermectin is more particularly an anthelmintic. It has been administered in humans in the treatment of onchocerciasis caused by onchocerca volvulus, of gastrointestinal strongyloidiasis (anguillulosis) (product Stomectol®) and of human scabies (Meinking T L et al., N. Engl. J. Med., 1995 Jul. 6; 333(1): 26-30 The treatment of scabies with ivermectin) and also in the treatment of microfilaremia diagnosed or suspected in individuals suffering from lymphatic filariosis due to Wuchereria bancrofti.

U.S. Pat. No. 4,199,569, discloses ivermectin as a semisynthetic, anthelmintic agent derived from the avermectins, a class of highly active broad-spectrum anti-parasitic agents isolated from the fermentation products of Streptomyces avermitilis.

U.S. Pat. Nos. 6,399,652; 6,399,651 and 6,319,945 disclose methods of treating skin disorders via application of topical formulations containing ivermectin to treat acne vulgaris (the '652 patent), a variety of dermatoses (e.g., transient acantholytic dermatitis, acne miliaris necrotica, acne varioliformis, perioral dermatitis, and acneiform eruptions; the '651 patent) and seborrheic dermatitis (the '945 patent).

PCT Application Pub. No. WO 2004/093886 discloses the use of ivermectin for the manufacture of a topical pharmaceutical composition intended for human use. More particularly, this application discloses the use of topical pharmaceutical compositions intended for human use comprising ivermectin for the treatment of dermatological conditions such as rosacea, acne vulgaris, seborrhoeic dermatitis, perioral dermatitis, acneform rash, transient acantholytic dermatitis and acne miliaris necrotica.

U.S. Pat. No. 5,952,372 describes a method for treating rosacea using ivermectin orally or topically in order to reduce and eliminate the parasite Demodex folliculorum present on the skin of patients.

U.S. Pat. Nos. 7,550,440, 8,080,530, 8,093,219, 8,415,311, 8,470,788 and 8,815,816 disclose a topical pharmaceutical oil-in-water type emulsion of ivermectin comprising an oily phase, a surfactant-emulsifier, a mixture of solvent and/or pro-penetrating agents for ivermectin and gelling agents. A commercialized product of ivermectin based on these patents is recently been launched under the brand name Soolantra® by Galderma Laboratories.

U.S. Pat. No. 8,815,938 discloses a skin care product containing a first anhydrous composition comprising an avermectin compound and a second composition. The two compositions are provided in separate compartments, a first anhydrous compartment and a second aqueous compartment. The patent further discloses that either of the two compartments is in the form of gel. The patent teaches that the product avoids the degradation of avermectin in contact with water or in contact with raw materials capable of degrading avermectin.

PCT Application Pub. No. WO 2005/089806 discloses a pharmaceutical composition based on a compound of the avermectin family, in the form of a cream-gel comprising, in a physiologically acceptable medium, an oily phase dispersed in an aqueous phase by means of a non-surfactant polymeric emulsifier.

U.S. Pat. Application Pub. No. 2008/0027011 discloses an oral pharmaceutical or veterinary paste or gel formulation which may comprise an effective amount of a therapeutic agent, optionally an absorbent or a viscosity modifier, optionally a hydrophilic carrier, a colorant, stabilizer, surfactant, or preservative and optionally an antioxidant, solvent, flavouring, buffering system or thickener and methods of preparing these formulations. The exemplified gel formulations contain antioxidants, preservatives and buffering systems along with other additives.

U.S. Pat. No. 7,064,108 discloses a topical gel composition of ivermectin comprising alcohol, glycol. The composition is useful for treating a Pediculosis capitis infestation in human.

The low compatibility of ivermectin with many excipients (N. O. Shaw, M. M. de Villiers and A. P. Lotter, Pharmazie, 54 (1999) 5, 372-376 Preformulation stability screening of ivermectin with non-ionic emulsion excipients), and its low solubility in water mean that pharmaceutical compositions containing ivermectin generally require either the addition of a large number of additives which make it possible to obtain stable compositions, which has the effect of increasing the risk of allergies, or to be formulated with anhydrous excipients. The anhydrous compositions encountered conventionally have the disadvantage of a greasy feel and therefore of an appearance that is not very cosmetic, which may be responsible for a decrease in patient compliance. In addition, by virtue of the low stability of ivermectin in water, the shelf life of aqueous compositions containing ivermectin is generally shorter than that of anhydrous compositions containing ivermectin.

Various methods have been provided in the prior art to stabilize ivermectin in aqueous compositions and overcome its chemically instability on contact with water.

European Pat. No. EP 0,045,655 B1 proposes forming micelles of surfactants which surround the ivermectin in order to protect it against water; other applications, such as PCT Application Pub. Nos. WO 01/60380 and WO 97/26895, propose using aqueous solvents for active agents, such as N-methyl-2-pyrrolidone. Finally, PCT Application Pub. Nos. WO 2004/093886 and WO 2005/089806 describe emulsions comprising an oily phase and an aqueous phase, said aqueous phase comprises a micellar active phase containing ivermectin.

There still exists a need for topical pharmaceutical compositions containing ivermectin which are completely suited to the pathological condition and specifically to sensitive skin, which is industrially acceptable, i.e., the formulation of which is physically stable (without phase separation) and chemically stable (without modification of the stability of the active agent), and which optimizes the penetration of ivermectin into the skin was therefore required.

Despite the various methods and formulations suggested in the art, most of the methods do not allow optimum stability of the ivermectin in the formulation. Moreover, the formulations which are believed to maintain chemical and physical stability of ivermectin requires a large number of ingredients in order to manufacture such formulations.

Therefore, there still exists a need to provide an alternative, chemically and physically stable and effective topical formulation, particularly a gel formulation of ivermectin which contains relatively less number of additives (or have minimum additive load), exhibits good physical and chemical stability and good tolerance on the skin.

### SUMMARY OF THE INVENTION

The present invention provides a topical gel composition of ivermectin. The composition is in the form of an aqueous gel composition comprising a pharmaceutically acceptable glycol and a pharmaceutically acceptable gelling agent. Essentially, the gel is devoid of alcohols.

In one aspect, the invention provides a topical aqueous gel composition comprising:
(a) Ivermectin;
(b) one or more pharmaceutically acceptable glycols;
(c) one or more pharmaceutically acceptable gelling agents; and
(d) water;
   wherein said gel composition is devoid of an alcohol.

In another aspect, the invention provides a topical aqueous gel composition comprising:
(a) Ivermectin;
(b) one or more pharmaceutically acceptable glycols selected from the group consisting of propylene glycol, 1,3-butylene glycol, polyethylene glycol, methylpropanediol, ethoxydiglycol, hexylene glycol, and dipropylene glycol;
(c) one or more pharmaceutically acceptable gelling agents selected from the group consisting of hydroxypropyl cellulose, hydroxypropyl methylcellulose, carrageenans, microcrystalline cellulose, alginates, gellan gum, xanthan gum, veegum, hydroxyethyethylcellulose, guar gum, and carbomers; and
(d) water;
   wherein said gel composition is devoid of an alcohol.

In another aspect, the invention provides a topical aqueous gel composition consisting essentially of:
(a) Ivermectin;
(b) one or more pharmaceutically acceptable glycols comprising propylene glycol;
(c) one or more pharmaceutically acceptable gelling agents comprising hydroxypropyl methylcellulose; and
(d) water;
   wherein said gel composition is devoid of an alcohol.

In another aspect, the amount of glycol in the topical aqueous gel composition of the invention is at least 45% by weight of the composition.

In another aspect, the invention provides a topical aqueous gel composition comprising:
(a) Ivermectin in an amount of about 0.01% to about 5% by weight;
(b) one or more pharmaceutically acceptable glycols in an amount of at least 45% by weight;
(c) one or more pharmaceutically acceptable gelling agents in an amount of about 0.01% to about 5% by weight; and
(d) water;
   wherein said gel composition is devoid of an alcohol.

In another aspect, the topical aqueous gel composition of ivermectin of the invention remains chemically and/or physically stable over a period of time of at least 8 weeks.

In another aspect, the topical aqueous gel composition of ivermectin of the invention further comprises one or more additives selected from the group consisting of flavour enhancers, preserving agents, stabilizers, humidity regulators, pH regulators, osmotic pressure modifiers, UV-A screening agents, UV-B screening agents and antioxidants. Essentially, the topical gel composition of ivermectin of the invention is devoid of any preserving agents and antioxidants.

In another aspect, the invention also provides a method for the treatment of rosacea, more particularly, for the treatment of inflammatory lesions of rosacea comprising topically applying the topical aqueous gel composition of ivermectin as substantially described herein above on to the affected skin of the patient in need of such treatment.

Still other aspects and advantages of the invention will be apparent from the following detailed description of the invention.

### DETAILED DESCRIPTION OF THE INVENTION

The invention provides for a topical gel composition of ivermectin with minimum additives load. It was surprisingly found that the composition according to the invention exhibits good stability and good tolerance on the skin. Particularly, it was found that such a stable composition can be prepared without using any alcohol. Such formulations are also easy to manufacture on a commercial scale.

The gel composition of the present invention provides a means for topically delivering ivermectin to a patient over a long residence time on the skin. This is particularly important since ivermectin is a slow acting drug. By providing a long residence time, the cosmetically acceptable gel formulation allows for therapeutically effective use of environmentally acceptable low quantities of ivermectin. Additionally, use of low quantities of ivermectin may reduce human systemic absorption of the active ingredient.

The ivermectin according to the invention may contain at least 80% of 22,23-dihydroavermectin B₁ₐ and less than 20% of 22,23-dihydroavermectin B_{1b}.

The topical gel composition of ivermectin according to the invention comprises:
(a) Ivermectin;
(b) one or more pharmaceutically acceptable glycols;
(c) one or more pharmaceutically acceptable gelling agents; and
(d) water.

The prior art gel formulations of ivermectin containing alcohol may cause drying of the skin. Essentially the topical gel composition of the invention is devoid of alcohol, particularly alcohols selected from methanol, benzyl alcohol, ethyl alcohol and isopropyl alcohol.

The topical gel composition of the invention is suitable for treating rosacea and more particularly for treating inflammatory lesions of rosacea.

Pharmaceutically acceptable glycols used in topical gel compositions of the invention are non-toxic, and do not irritate the skin at the concentrations. Suitable glycols include, but are not limited to, propylene glycol such as propylene glycol 200 (PEG 200), 1,3-butylene glycol, polyethylene glycol 400 (PEG 400), methylpropanediol, ethoxydiglycol (e.g. Transcutol®), hexylene glycol, and dipropylene glycol. Propylene glycol is preferred.

The amount in which pharmaceutically acceptable glycols are present in the topical gel compositions of the invention is at least 45% by weight of the composition.

Pharmaceutically acceptable gelling agents suitable for use in the topical gel composition of the invention are those agents which provide viscosity to a formulation such that the formulation can be effectively applied to the infested or affected area, including but not limited to hydroxypropylcellulose (e.g. Klucel H A), hydroxypropyl methylcellulose carrageenans, microcrystalline cellulose, carbomer, alginates, gellan gum, xanthan gum, veegum, hydroxyethyethyl cellulose, guar gum and carbomers. Hydroxypropyl methylcellulose is preferred.

The topical gel composition according to the invention comprise from 0.001% to 10% of ivermectin by weight relative to the total weight of the composition. Preferably, the composition according to the invention contains from 0.1% to 5% of ivermectin by weight relative to the total weight of the composition.

The amount in which pharmaceutically acceptable gelling agents are present in the topical gel compositions of the invention ranges from about 0.01% to about 5% by weight of the composition.

The composition according to the invention is advantageously an aqueous gel which comprises:
(a) Ivermectin in an amount of about 0.01% to about 5% by weight;
(b) one or more pharmaceutically acceptable glycols in an amount of at least 45% by weight;
(c) one or more pharmaceutically acceptable gelling agents in an amount of about 0.01% to about 5% by weight; and
(d) water.

The topical gel composition of the invention contains water ranging from 1% to 45% by weight relative to the total weight of the composition. The water used in the composition according to the invention will preferably be purified water.

The topical gel composition according to the invention optionally contains non-ionic surfactants. Suitable non-ionic surfactants can be selected from the group consisting of Cetostearyl alcohol, Cetyl alcohol, Cocamide DEA, Cocamide MEA, Isoceteth-20, Oleyl alcohol, Sorbitan monostearate, Sorbitan tristearate, Stearyl alcohol, tyloxapol, softigen, solutol HS15, poloxamers such as Pluronic F-68LF™ or Lutrol F68, Pluronic L-62LF™ and Pluronic L62D™ (BASF Wyandotte Corp., Parsippany, N.J., USA), polysorbates such as polysorbate 20 and polysorbate 80, polyoxyethylene fatty acid esters such as Emulphor™ (GAF Corp., Wayne, N.J., USA). The topical gel composition may comprise up to 15% by weight of suitable surfactant(s), preferably from about 5% to about 15% by weight of the composition.

The compositions according to the invention are described as a stable gel composition in that it may exhibit good physical and chemical stability over time, even at a temperature above ambient temperature (for example 45-55° C.).

The ivermectin in the topical gel composition according to the invention also, surprisingly, may exhibit good chemical stability in the case of pH variation.

The topical gel composition according to the invention may also contain inert additives or combinations of these additives, such as flavour enhancers; preservatives; stabilizers; humidity regulators; pH regulators; osmotic pressure modifiers; UV-A and UV-B screening agents; and antioxidants. Preferably, the topical gel composition of ivermectin of the invention is devoid of preserving agents and antioxidants.

These additives may be present in the composition at from 0.001% to 20% by weight relative to the total weight of the composition.

A typical gel composition according to the invention comprises:
(a) Ivermectin;
(b) one or more pharmaceutically acceptable glycols comprising propylene glycol;
(c) one or more pharmaceutically acceptable gelling agents comprising hydroxypropyl methylcellulose; and
(d) water.

The pH of the composition preferably ranges from 5.0 to 8.0. Verification of the natural pH of the mixture and possible correction with a solution of a neutralizing agent, and also the incorporation of the optional additives, may be carried out, according to their chemical nature, during one of the steps of the method of preparation, described above.

Preparation of the gel composition of the invention involves the sequential or separate mixing of ivermectin in the pharmaceutically acceptable glycol, the pharmaceutically acceptable gelling agent, and water. Additional optional additives such as non-ionic surfactant may be added with ivermectin.

A preferred preparation involves the mixing of ivermectin in propylene glycol, separate mixing of hydroxypropyl methylcellulose in water and mixing of the two mixtures together for form an aqueous gel.

This invention also features the topical gel composition according to the invention as pharmaceutical preparations useful to treat dermatological conditions/afflictions.

The formulation of ivermectin into a topical gel composition for human use according to the invention is particularly useful for the treatment of rosacea, of common acne, of seborrhoeic dermatitis, of perioral dermatitis, of acneform rashes, of transient acantholytic dermatosis, and of acne necrotica miliaris.

The formulation of ivermectin into topical gel composition for human use according to the invention is more particularly useful in a regime or regimen for the treatment of rosacea.

### EXAMPLE 1

### Ivermectin Gel

TABLE 1

| Sr. | | Quantity | Quantity | Quantity |
|---|---|---|---|---|
| No. | Ingredients | (% w/w) | (% w/w) | (% w/w) |
| 1 | Ivermectin | 1.00 | 2.00 | 3.00 |
| 2 | Propylene Glycol | 53.00 | 54.00 | 55.00 |
| 3 | Hypromellose | 1.00 | 1.00 | 1.00 |
| 4 | Purified Water | QS | QS | QS |

Process: Ivermectin was dissolved in propylene glycol. Separately, hypromellose was dispersed in water by high shear mixing. The ivermectin solution and hypromellose dispersion were then mixed together until a gel was formed. Any entrapped air was removed by deaeration. The gel was filled and packed in laminate/aluminium tubes.

What is claimed is:

1. A topical aqueous gel composition comprising:
(a) Ivermectin;
(b) one or more pharmaceutically acceptable glycols;
(c) one or more pharmaceutically acceptable gelling agents; and
(d) water;
wherein said gel composition is devoid of alcohol.

2. The gel composition of claim 1, wherein the ivermectin is present in an amount of about 0.01% to about 5% by weight of said gel composition.

3. The gel composition of claim 1, wherein the pharmaceutically acceptable glycol is present in an amount of at least 45% by weight of said gel composition.

4. The gel composition of claim 1, wherein the pharmaceutically acceptable gelling agent is present in an amount of at about 0.1% to about 5% by weight of said gel composition.

5. The gel composition of claim 1, wherein the glycol is selected from the group consisting of propylene glycol, 1,3-butylene glycol, polyethylene glycol, methylpropanediol, ethoxydiglycol, hexylene glycol, and dipropylene glycol.

6. The gel composition of claim 1, wherein the gel composition remains chemically and/or physically stable over a period of time of at least 8 weeks.

7. The gel composition of claim 1, wherein said gel composition further comprises one or more additives selected from the group consisting of flavour enhancers, preserving agents, stabilizers, humidity regulators, pH regulators, osmotic pressure modifiers, UV-A screening agents, UV-B screening agents and antioxidants.

8. The gel composition of claim 1, wherein said gel composition is devoid of preserving agents and antioxidants.

9. The gel composition of claim 1, wherein said get composition is devoid of an alcohol selected from the group consisting of methanol, benzyl alcohol, ethyl alcohol and isopropyl alcohol.

10. A method for the treatment of rosacea or inflammatory lesions of rosacea comprising topically applying the gel composition of claim 1 on to the affected skin of the patient in need of such treatment.

11. A topical aqueous gel composition comprising:
(a) ivermectin;
(b) one or more pharmaceutically acceptable glycols selected from the group consisting of propylene glycol, 1,3-butylene glycol, polyethylene glycol, methylpropanediol, ethoxydiglycol, hexylene glycol, and dipropylene glycol;
(c) one or more pharmaceutically acceptable gelling agents selected from the group consisting of hydroxypropyl cellulose, hydroxypropyl methylcellulose, carrageenans, microcrystalline cellulose, alginates, gellan gum, xanthan gum, veegum, hydroxyethyethylcellulose, guar gum, and carbomers; and
(d) water;
wherein said gel composition is devoid of alcohol.

12. The topical aqueous gel composition of claim 11, wherein the gel composition is devoid of an alcohol selected from the group consisting of methanol, benzyl alcohol, ethyl alcohol and isopropyl alcohol.

13. A topical aqueous gel composition consisting essentially of:
(a) ivermectin;
(b) one or more pharmaceutically acceptable glycols comprising propylene glycol;
(c) one or more pharmaceutically acceptable gelling agents comprising hydroxypropyl methylcellulose; and
(d) water;
wherein said gel composition is devoid of alcohol.

14. The topical aqueous gel composition of claim 13, wherein the composition consists of:
(a) ivermectin;
(b) propylene glycol;
(c) hydroxypropyl methylcellulose; and
(d) water;
wherein said gel composition is devoid of alcohol.

15. The topical aqueous gel composition of claim 14, wherein the composition consists of:
(a) about 0.01% to about 5% by weight of ivermectin;
(b) at least 53-55% by weight of propylene glycol;
(c) about 0.1% to about 5% by weight of hydroxypropyl methylcellulose; and
(d) water;
wherein said gel composition is devoid of alcohol.

16. The topical aqueous gel composition of claim 14, wherein the composition consists of:
(a) about 1% to about 5% by weight of ivermectin;
(b) about 53-55% by weight of propylene glycol;
(c) about 1% by weight of hydroxypropyl methylcellulose; and
(d) water;
wherein said gel composition is devoid of alcohol.

17. The topical aqueous gel composition of claim 13, wherein the gel composition is devoid of an alcohol selected from the group consisting of methanol, benzyl alcohol, ethyl alcohol and isopropyl alcohol.

18. The topical aqueous gel composition of claim 13, wherein the gel composition is devoid of preserving agents and antioxidants.
